SEQUENCE LISTING (17 sequences)

SEQ ID NO: 1 - DNA, 513 bp, Triatoma protracta
  CDS (1)..(513)
atgaagacgttcattgtaatcacatttattggaatcctgtcctacgcatatgctgcaaacgaatgtgaaaatccagaacctatgcaaggttttagtgcctcacaattttaccaaggaaagtggtatgtgactcatgaaacatccgcatcaactctatccgaatgtaacattcttactactagcaatgataatggcaaattcactgttaaacataaatataccaaagatggaaaagtgggtgaactcatttgtgagggtcaagcaagcgcaaataataaatttacttacgactgcaaatttgacggagagactatggagcaagtaactcgaacagcaatggatacagactataatgactacgctctgtactatttatgtacaacttataaaaatggtcctaatgctggcaagaaagaagggcattatattctttcaagaagacaaccaaatacagaaattcctgatgcgctgaaaactaagacaaaggatttgaatttgaaattatgtggttaa

SEQ ID NO: 2 - protein, 170 aa, Triatoma protracta
MKTFIVITFIGILSYAYAANECENPEPMQGFSASQFYQGKWYVTHETSASTLSECNILTTSNDNGKFTVKHKYTKDGKVGELICEGQASANNKFTYDCKFDGETMEQVTRTAMDTDYNDYALYYLCTTYKNGPNAGKKEGHYILSRRQPNTEIPDALKTKTKDLNLKLCG

SEQ ID NO: 3 - DNA, 513 bp, Triatoma protracta
  CDS (1)..(513)
atgaagacgttcattgtaatcacatttattggaatcctgtcctacgcatatgctgcaaacgaatgtgaaaatccaaaacctatggaaggttttagtgcctcacaattttaccaaggaaagtggtatgtgactcatgaaacatccgcatcaactctatccgaatgtaacattcttactactagcaatgataatggcaaattcactgttaaacataaatataccaaagatggaaaagtgggtgaactcatttgtgagggtcaagcaagcgcaaataataaatttacttacgactgcaaatttgacggagagactatggagcaagtaactcgaacagcaatggatacagactataatgactacgctctgtactatttatgtacaacttataaaaatggtcctaatgctggcaagaaagaagggcattatattctttcaagaagaaaaccaaatacagaaattcctgatgcgctgaaaactaagacaaaggatttgaatttgaaaatatgtggttaa

SEQ ID NO: 4 - protein, 170 aa, Triatoma protracta
MKTFIVITFIGILSYAYAANECENPKPMEGFSASQFYQGKWYVTHETSASTLSECNILTTSNDNGKFTVKHKYTKDGKVGELICEGQASANNKFTYDCKFDGETMEQVTRTAMDTDYNDYALYYLCTTYKNGPNAGKKEGHYILSRRKPNTEIPDALKTKTKDLNLKICG

SEQ ID NO: 5 - protein, 5 aa, Artificial Sequence
  Peptide Linker
GGGGS

SEQ ID NO: 6 - protein, 12 aa, Artificial Sequence
  Peptide Linker
GKSSGSGSESKS

SEQ ID NO: 7 - protein, 14 aa, Artificial Sequence
  Peptide Linker
GSTSGSGKSSEGKG

SEQ ID NO: 8 - protein, 18 aa, Artificial Sequence
  Peptide Linker
GSTSGSGKSSEGSGSTKG

SEQ ID NO: 9 - protein, 18 aa, Artificial Sequence
  Peptide Linker
GSTSGSGKPGSGEGSTKG

SEQ ID NO: 10 - protein, 14 aa, Artificial Sequence
  Peptide Linker
EGKSSGSGSESKEF

SEQ ID NO: 11 - protein, 5 aa, Artificial Sequence
  Peptide Localization Sequence
KKKRK

SEQ ID NO: 12 - protein, 26 aa, Artificial Sequence
  Peptide Localization Sequence
MLRTSSLFTRRVQPSLFRNILRLQST

SEQ ID NO: 13 - protein, 4 aa, Artificial Sequence
  Peptide Localization Sequence
KDEL

SEQ ID NO: 14 - protein, 4 aa, Artificial Sequence
  Peptide Localization Sequence
CAAX

SEQ ID NO: 15 - protein, 4 aa, Artificial Sequence
  Peptide Localization Sequence
CCXX

SEQ ID NO: 16 - DNA, 18 bp, Artificial Sequence
  Oligonucleotide Primer
cctgtcctacgcatatgc

SEQ ID NO: 17 - DNA, 21 bp, Artificial Sequence
  Oligonucleotide Primer
gcataggttctggattttcac

(19) United States
(12) Patent Application Publication
(10) Pub. No.: US 2010/0239586 A1
(43) Pub. Date: Sep. 23, 2010

(54) PROCALIN POLYPEPTIDES, ANTIGENS, AND METHODS OF USE

(75) Inventors: David D. Lo, Del Mar, CA (US); Mandy Sze Man Wong, Chino Hills, CA (US); Christiane Weirauch, Riverside, CA (US); Ryan C. Smith, Anaheim, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/729,203

(22) Filed: Mar. 22, 2010

Related U.S. Application Data
(60) Provisional application No. 61/162,268, filed on Mar. 21, 2009.

(51) Int. Cl. A61K 39/395; A61K 39/00; G01N 33/53; A61K 38/17; C07K 14/435; C07K 16/18; C07H 21/04

(52) U.S. Cl. 424/139.1; 424/185.1; 435/7.1; 514/12; 530/350; 530/387.9; 536/23.5

(57) ABSTRACT

The disclosure provide polypeptides and polynucleotides encoding Triatoma sp. antigens and methods of using such polypeptides and polynucleotides.

### CROSS REFERENCE TO RELATED APPLICATIONS

This application claims priority under 35 U.S.C. §119 from Provisional Application Ser. No. 61/162,268, filed Mar. 21, 2009, the disclosure of which is incorporated herein by reference.

### FIELD OF THE DISCLOSURE

The invention relates to polynucleotides and polypeptides, antibodies to such polypeptides, vaccines and methods of diagnosis and treatment.

### BACKGROUND

Many inflammatory and anaphylactic reactions to biting insects can be attributable to a small but important group of hematophagous bugs that comprise the subfamily Triatominae (Heteroptera:Reduviidae).

### SUMMARY

The disclosure provides a substantially purified procalin 1 polypeptide, comprising: (i) a polypeptide encoded by SEQ ID NO:1, (ii) a polypeptide comprising SEQ ID NO:2, (iii) a polypeptide having at least 80%, 90%, 95%, 98%, 99% identity to SEQ ID NO:2 and having anticoagulant effect, vasodilatoryeffect or a combination thereof, (iv) a polypeptide comprising SEQ ID NO:2 and having at least 1-30 (e.g., 1-10, 5-15, 10-20, or 15-30) conservative amino acid substitutions and having anticoagulant effect, vasodilatory effect or a combination thereof, (v) a polypeptide having at least 80%, 90%, 95%, 98%, 99% identity to SEQ ID NO:2 and having a Glu at position 26, a Gln at position 29, a Gln at position 148, a Leu at position 168, and any combination thereof and having anticoagulant effect, vasodilatoryeffect or a combination thereof, (vi) a polypeptide comprising SEQ ID NO:2 and having at least 1-30 (e.g., 1-10, 5-15, 10-20, or 15-30) conservative amino acid substitutions, and having a Glu at position 26, a Gln at position 29, a Gln at position 148, a Leu at position 168, and any combination thereof and having anticoagulant effect, vasodilatoryeffect or a combination thereof, or (vii) a polypeptide comprising a fragment of any of the foregoing wherein an antibody raised against the fragment specifically binds to a procalin 1 polypeptide.

The disclosure also provides a substantially purified procalin 2 polypeptide, comprising: (i) a polypeptide encoded by SEQ ID NO:3, (ii) a polypeptide comprising SEQ ID NO:4, (iii) a polypeptide having at least 80%, 90%, 95%, 98%, 99% identity to SEQ ID NO:4 and having anticoagulant effect, vasodilatoryeffect or a combination thereof, (iv) a polypeptide comprising SEQ ID NO:4 and having at least 1-30 (e.g., 1-10, 5-15, 10-20, or 15-30) conservative amino acid substitutions and having anticoagulant effect, vasodilatory effect or a combination thereof, (v) a polypeptide having at least 80%, 90%, 95%, 98%, 99% identity to SEQ ID NO:4 and having a Lys at position 26, a Glu at position 29, a Lys at position 148, a Iso at position 168, and any combination thereof and having anticoagulant effect, vasodilatoryeffect or a combination thereof, (vi) a polypeptide comprising SEQ ID NO:4 and having at least 1-30 (e.g., 1-10, 5-15, 10-20, or 15-30) conservative amino acid substitutions, and having a Lys at position 26, a Glu at position 29, a Lys at position 148, a Iso at position 168, and any combination thereof and having anticoagulant effect, vasodilatoryeffect or a combination thereof, or (vii) a polypeptide comprising a fragment of any of the foregoing wherein an antibody raised against the fragment specifically binds to a procalin 1 polypeptide.

The disclosure further provides a polynucleotide encoding a procalin polypeptide of the disclosure. In one aspect, the polynucleotide comprises (i) a polynucleotide encoding a polypeptide comprising SEQ ID NO:2 or 4; (ii) a polynucleotide comprising SEQ ID NO:1 or 3; (iii) a polynucleotide that hybridizes to a nucleic acid consisting of SEQ ID NO:1 or 3 and which encodes a polypeptide having procalin activity (e.g., antihemostatic, anticoagulant or vasodilation effects); (iv) a polynucleotide that is substantially identical to SEQ ID NO:1 or 3 and encodes a polypeptide having procalin activity (e.g., antihemostatic, anticoagulant or vasodilation effects); (v) a polynucleotide that comprises a sequence that is complementary to SEQ ID NO:1 or 3; (vi) a polynucleotide or oligonculeotide fragment of any of the foregoing encoding an antigenic fragment of a procalin polypeptide and (vii) any of the foregoing wherein T can be U.

The disclosure also provides a composition comprising a polypeptide or polynucleotide of the disclosure in a pharmaceutically acceptable carrier.

The disclosure also provides an immunogenic composition comprising a procalin polypeptide, allergen, or antigenic epitope of the disclosure and a pharmaceutically acceptable carrier. In one aspect, the composition may comprise an adjuvant.

The disclosure also provides a method of desensitizing a subject to a Triatoma antigen comprising contacting the subject with a composition of the disclosure such that an immunological reaction is elicited.

The disclosure also provides a substantially purified antibody that specifically binds to a procalin variant 1 or procalin variant 2. In some aspect, the antibody may be prepared in a composition comprising a pharmaceutically acceptable carrier.

The disclosure also provides a method of identifying a source of an anaphylactic reaction comprising contacting a sample from a subject having an anaphylactic reaction with (i) a polypeptide or peptide comprising a sequence as set forth in SEQ ID NO:2 or 4, or (ii) an antibody that specifically binds to a polypeptide produced by a polynucleotide comprising a sequence as set forth in SEQ ID NO:1 or 3.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 shows the polynucleotide and polypeptide sequence of a procalin 1 variant of the disclosure.

FIG. 2 shows the polynucleotide and polypeptide sequence of a procalin 2 variant of the disclosure.

FIG. 3 shows a blot depicting an isolated procalin variant of the disclosure.

### DETAILED DESCRIPTION

As used herein and in the appended claims, the singular forms “a,” “and,” and “the” include plural referents unless the context clearly dictates otherwise. Thus, for example, reference to “a polypeptide” includes a plurality of such polypeptides and reference to “the protein” includes reference to one or more proteins, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Although methods and materials similar or equivalent to those described herein can be used in the practice of the disclosed methods and compositions, the exemplary methods and materials are described herein.

Unless defined otherwise, all technical and scientific terms used herein have the same meaning as commonly understood to one of ordinary skill in the art to which this disclosure belongs. Thus, as used throughout the instant application, the following terms shall have the following meanings.

The publications discussed above and throughout the text are provided solely for their disclosure prior to the filing date of the present application. Nothing herein is to be construed as an admission that the inventors are not entitled to antedate such disclosure by virtue of prior disclosure.

Many inflammatory and anaphylactic reactions to biting insects can be attributable to a small but important group of hematophagous bugs that comprise the subfamily Triatominae (Heteroptera:Reduviidae). Although best known as arthropod vectors of Chagas disease, these insects also inject salivary proteins during the acquisition of blood from a subject that may initiate a variety of severe and occasionally fatal allergic responses in sensitized individuals. Systemic reactions include generalized pruritis, gastrointestinal disturbances, fever, dyspnea, syncope, hypotension, laryngeal and glossal edema, and convulsions. Extreme hypersensitivity resulting in death has been attributed to the bite of Triatoma protracta. In the U.S., allergic reactions have been associated with T. protracta, T. rubida, T. recurva, T. sanguisuga, T. gerstaekeri, and Paratriatoma hirsuta. Allergic sensitization, demonstrated by anti-Triatoma IgE Ab, may develop in as many as 7% of individuals residing within the range of these insects. The expansion in both seasonal and perennial human incursions into chaparral or woodland habitats of T. protracta in the western U.S. has increased the number of persons at risk for Triatoma hypersensitivity; it is estimated that greater than 30,000 persons in California are at risk for anaphylaxis from this insect.

Little is known about the molecular identity of salivary allergens of these insects. Initial studies with T. protracta indicate that ˜89% of the allergenic activity in the saliva of this bug represents reaction to an 18- to 20-kDa protein. The disclosure provides variants of procalin having antigenic activity and which are useful in diagnostics, risk assessment, desensitization therapeutics, and vaccines. The purification of these allergenic protein and isolation of a cDNA clones are useful for the generation of desensitization therapeutics, vaccines, in diagnostics and risk assessment. For examples, successful expression of recombinant antigen in yeast or other appropriate expression systems provides reagent quantities of antigen for subsequent investigations of the serologic diagnosis, epidemiology, and desensitization therapy of subjects at risk of severe allergic reaction to the bite of Triatoma sp. (e.g., T. protracta). In some embodiments, a procalin polypeptide can be used for therapy where vasodilatation or anticoagulation therapy is needed.

Procalin belongs to a family of lipocalin proteins, which has been functionally associated with anti-hemostatic activity, anticoagulation, and induction of vasodilation. The disclosure provides sequences of procalin derived from wild specimens, and recombinant proteins encoded by these sequences.

The term “antigen”, as used herein, refers to a molecule that elicits production of an antibody and/or an antigen-specific reaction with T-cells in an animal.

The term “allergen”, as used herein, refers to a subset of antigens which elicit the production of IgE antibodies. The polypeptides of the disclosure are allergens.

An “allergic reaction”, as used herein, is an immune response that is IgE mediated with clinical symptoms primarily involving the cutaneous (e.g., uticana, angiodema, pruritus), respiratory (e.g., wheezing, coughing, laryngeal edema, rhinorrhea, watery/itching eyes), gastrointestinal (e.g., vomiting, abdominal pain, diarrhea) and cardiovascular (i.e., if a systemic reaction occurs) systems.

An “anaphylactic allergen”, refers to a subset of allergens that are recognized to present a risk of anaphylactic reaction in allergic individuals when encountered in its natural state.

“Anaphylaxis” or an “anaphylactic reaction” refers to a subset of allergic reactions characterized by mast cell degranulation secondary to cross-linking of the high-affinity IgE receptor on mast cells and basophils induced by an anaphylactic allergen with subsequent mediator release and the production of severe systemic pathological responses in target organs. As is known in the art, the severity of an anaphylactic reaction may be monitored, for example, by assaying cutaneous reactions, puffiness around the eyes and mouth, vomiting and/or diarrhea, followed by respiratory reactions such as wheezing and labored respiration. The most severe anaphylactic reactions can result in loss of consciousness and/or death.

An “antigen presenting cell” or “APC”, refers to a cell which processes and presents antigens to T-cells to elicit an antigen-specific response, e.g., macrophages and dendritic cells.

An “epitope,” refers to a binding site including an amino acid motif of between approximately six and fifteen amino acids which can be bound by an immunoglobulin (e.g., IgE, IgG, etc.) or recognized by a T-cell receptor when presented by an APC in conjunction with the major histocompatibility complex (MHC). A linear epitope is one where the amino acids are recognized in the context of a simple linear sequence. These linear epitopes are also commonly referred to as sequential epitopes in the art. A conformational epitope is one where the amino acids are recognized in the context of a particular three dimensional structure.

An antigen of the disclosure can comprise (a) a substantially, and in certain embodiments exactly, the same amino acid sequence as a naturally occurring procalin polypeptide or variant thereof and which is purified from a natural source (e.g., a purified polypeptide) or recombinantly produced polypeptide or peptide in a non-natural host. In certain embodiments, a recombinant allergen is produced in culture, typically in a unicellular host such as, for example, a bacterial host. In certain embodiments all immunodominant linear IgE epitopes within the protein allergen are preserved within a recombinant allergen. In certain embodiments, all non-immunodominant linear IgE epitopes are also preserved. In certain embodiments, a recombinant allergen may include the exact same amino acid sequence as a naturally occurring protein allergen. In other embodiments, a recombinant allergen may include substantially the same amino acid sequence. For example, the amino acid sequence can be at least 90%, 95%, or 99% identical to the sequence of the protein allergen or peptide containing epitope thereof. Typically any changes compared to a wild-type polypeptide will comprise conservative substitutions. In certain embodiments, a recombinant allergen may include one or more terminal amino acids that are absent from the naturally occurring protein allergen. In particular, terminal amino acids may be added to increase expression of the recombinant allergen, as a consequence of the vector used for expression, and the like. In addition, amino acid segments that are absent from the protein allergen may be added to the amino and/or carboxyl terminus of a recombinant allergen, e.g., tags for purification, labels for detection, tags that increase the solubility of the recombinant allergen, tags that increase the stability and/or bioavailability of the recombinant allergen, and the like. A proteolytic cleavage site may be introduced at the junction of the added amino acid segment and the recombinant allergen terminus to enable removal of the added segment after the recombinant allergen has been purified, absorbed, and the like. Common terminal modifications used in recombinant technology are described in Current Protocols in Molecular Biology Ed. by Ausubel et al., John Wiley & Sons, New York, N.Y., 1989 and Molecular Cloning: A Laboratory Manual Ed. by Sambrook et al., Cold Spring Harbor Press, Plainview, N.Y., 1989.

It will be appreciated that the amino acid sequence of a protein allergen encountered by an APC in vivo (i.e., within an exposed animal) may, in certain cases, differ from the full length amino acid sequence that is encoded by a cDNA clone of the naturally occurring protein allergen.

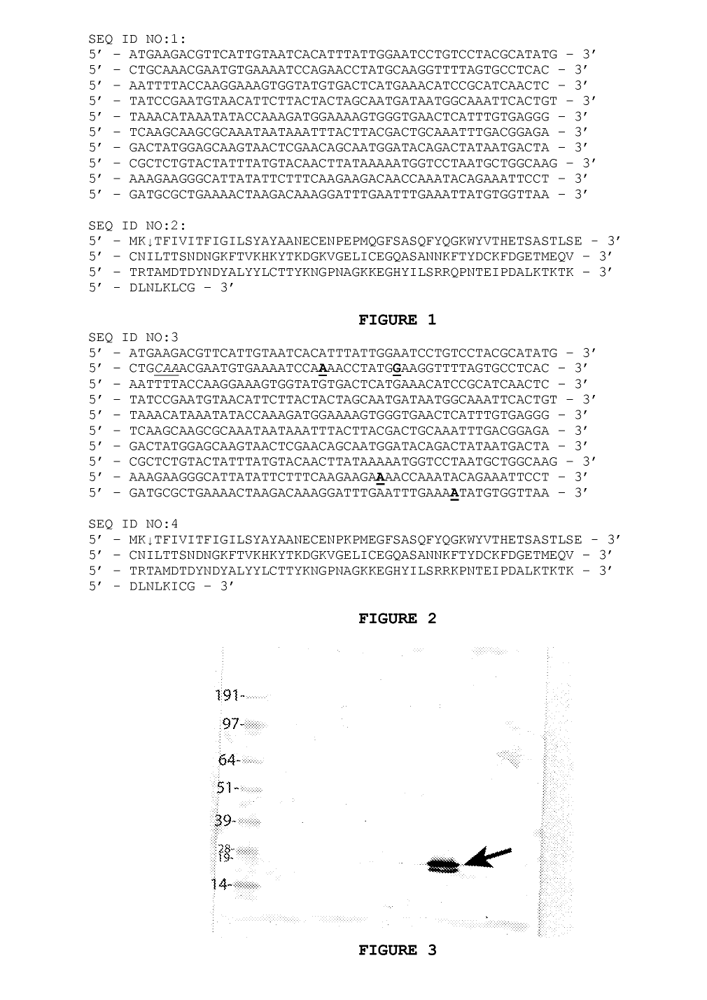

As used herein a “procalin polypeptide” means a polypeptide that contains or comprises an amino acid sequence as set forth in FIG. 1 or 2; polypeptides having substantial homology or substantial identity to the sequences set forth in SEQ ID Nos: 2 or 4; fragments of the foregoing sequences; and conservative variants of the foregoing. The disclosure provides polypeptides having a sequence as set forth in SEQ ID Nos: 2 or 4. In particular embodiments, a procalin polypeptide refers to a polypeptide comprising 80%, 85%, 90%, 95%, 98% or 99% identity to SEQ ID NOs: 2 or 4, except that residues 26, 29, 148 and 168 as set forth in SEQ ID NO:2 are unchanged. A procalin 1 polypeptide can be selected from the group consisting of (i) a polypeptide encoded by SEQ ID NO:1, (ii) a polypeptide comprising SEQ ID NO:2, (iii) a polypeptide having at least 80%, 90%, 95%, 98%, 99% identity to SEQ ID NO:2 and having anticoagulant effect, vasodilatoryeffect or a combination thereof, (iv) a polypeptide comprising SEQ ID NO:2 and having at least 1-30 (e.g., 1-10, 5-15, 10-20, or 15-30) conservative amino acid substitutions and having anticoagulant effect, vasodilatory effect or a combination thereof, (v) a polypeptide having at least 80%, 90%, 95%, 98%, 99% identity to SEQ ID NO:2 and having a Glu at position 26, a Gln at position 29, a Gln at position 148, a Leu at position 168, and any combination thereof and having anticoagulant effect, vasodilatoryeffect or a combination thereof, (vi) a polypeptide comprising SEQ ID NO:2 and having at least 1-30 (e.g., 1-10, 5-15, 10-20, or 15-30) conservative amino acid substitutions, and having a Glu at position 26, a Gln at position 29, a Gln at position 148, a Leu at position 168, and any combination thereof and having anticoagulant effect, vasodilatoryeffect or a combination thereof, and (vii) a polypeptide comprising a fragment of any of the foregoing wherein an antibody raised against the fragment specifically binds to a procalin 1 polypeptide. A procalin 2 polypeptide can be selected from the group consisting of (i) a polypeptide encoded by SEQ ID NO:3, (ii) a polypeptide comprising SEQ ID NO:4, (iii) a polypeptide having at least 80%, 90%, 95%, 98%, 99% identity to SEQ ID NO:4 and having anticoagulant effect, vasodilatoryeffect or a combination thereof, (iv) a polypeptide comprising SEQ ID NO:4 and having at least 1-30 (e.g., 1-10, 5-15, 10-20, or 15-30) conservative amino acid substitutions and having anticoagulant effect, vasodilatory effect or a combination thereof, (v) a polypeptide having at least 80%, 90%, 95%, 98%, 99% identity to SEQ ID NO:4 and having a Lys at position 26, a Glu at position 29, a Lys at position 148, a Iso at position 168, and any combination thereof and having anticoagulant effect, vasodilatoryeffect or a combination thereof, (vi) a polypeptide comprising SEQ ID NO:4 and having at least 1-30 (e.g., 1-10, 5-15, 10-20, or 15-30) conservative amino acid substitutions, and having a Lys at position 26, a Glu at position 29, a Lys at position 148, a Iso at position 168, and any combination thereof and having anticoagulant effect, vasodilatoryeffect or a combination thereof, and (vii) a polypeptide comprising a fragment of any of the foregoing wherein an antibody raised against the fragment specifically binds to a procalin 1 polypeptide.

Substantially identical sequences can be identified by those of skill in the art as having structural domains and/or having biological activity in common with an procalin. Methods of determining similarity or identity may employ computer algorithms such as, e.g., BLAST, FASTA, and the like. In one aspect, the disclosure comprises a polypeptide that is at least 80, 90, 92, 95, 98, or 99% identical to a polypeptide comprising SEQ ID NO:2 or 4 and having at residue 26 an E or K, respectively, at residue 29 a Q or E, respectively, at residue 148 a Q or K, respectively, at residue 168 and L or I, respectively.

Polypeptides derived from the procalins of the disclosure by any type of alteration (e.g., insertions, deletions, or substitutions of amino acids; changes in the state of glycosylation of the polypeptide; refolding or isomerization to change its three-dimensional structure or self-association state; and changes to its association with other polypeptides or molecules) are also encompassed by the disclosure. Therefore, the polypeptides provided by the disclosure include polypeptides characterized by amino acid sequences similar to those as set forth in FIG. 1 or 2, but into which modifications are naturally provided or deliberately engineered. A polypeptide that shares biological activities in common with a polypeptide comprising a sequence as set forth SEQ ID Nos: 2 or 4 having anticoagulant, vasodilation, antihemostatic effect characteristics or activity are encompassed by the disclosure. The disclosure also includes fragments of the polypeptides of the disclosure comprising at least 6-10 contiguous amino acid residues. Such fragments are useful for the generation of antibodies useful for diagnostic assays, purification assays and therapeutics. The disclosure encompasses various forms of procalin (e.g., procalin variant 1 (SEQ ID NO:2) and procalin variant 2 (SEQ ID NO:4)) and fragments thereof capable of illiciting an immune response.

The disclosure provides both full-length and mature forms of a procalin. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. The “mature form” of a polypeptide refers to a polypeptide that has undergone post-translational processing steps, if any, such as, for example, cleavage of the signal sequence or proteolytic cleavage to remove a prodomain. Multiple mature forms of a particular full-length polypeptide may be produced, for example, by imprecise cleavage of the signal sequence, or by differential regulation of proteases that cleave the polypeptide. The mature form(s) of such polypeptide may be obtained by expression, in a suitable insect or mammalian cell or other host cell, of a polynucleotide that encodes the full-length polypeptide. The sequence of the mature form of the polypeptide may also be determinable from the amino acid sequence of the full-length form, through identification of signal sequences or protease cleavage sites. The Procalins of the disclosure also include polypeptides that result from post-transcriptional or post-translational processing events such as alternate mRNA processing which can yield a truncated but biologically active polypeptide. Also encompassed within the disclosure are variations attributable to proteolysis such as differences in the N- or C-termini upon expression in different types of host cells, due to proteolytic removal of one or more terminal amino acids from the polypeptide (generally from 1-5 terminal amino acids).

A polypeptide of the disclosure may be prepared by culturing transformed or recombinant host cells under culture conditions suitable to express a procalin 1 or procalin 2 polypeptide of the disclosure. The resulting expressed polypeptide may then be purified from such culture using known purification processes. The purification of the polypeptide may also include an affinity column containing agents which will bind to the polypeptide; one or more column steps over such affinity resins as concanavalin A-agarose, heparin-Toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, the polypeptide of the disclosure may also be expressed in a form that will facilitate purification. For example, it may be expressed as a fusion polypeptide, such as those of maltose binding polypeptide (MBP), glutathione-S-transferase (GST) or thioredoxin (TRX). Kits for expression and purification of such fusion polypeptides are commercially available from New England BioLab (Beverly, Mass.), Pharmacia (Piscataway, N.J.), and InVitrogen, respectively. The polypeptide can also be tagged with an epitope and subsequently purified by using a specific antibody directed to such epitope. One or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous recombinant polypeptide. The polypeptide thus purified is substantially free of other mammalian polypeptides and is defined in accordance with the disclosure as a “substantially purified polypeptide”; such purified polypeptides include procalin, fragments, variants, and the like.

It is also possible to utilize an affinity column such as a monoclonal antibody generated against polypeptides of the disclosure, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the disclosure. In this aspect of the disclosure, proteins that bind a polypeptide of the disclosure (e.g., an anti-procalin variant 1 and anti-procalin variant 2 antibody of the disclosure) can be bound to a solid phase support or a similar substrate suitable for identifying, separating, or purifying cells that express polypeptides of the disclosure on their surface. Adherence of, for example, an anti-procalin variant 1 and procalin variant 2 antibody of the disclosure to a solid phase surface can be accomplished by any means, for example, magnetic microspheres can be coated with these polypeptide-binding proteins and held in the incubation vessel through a magnetic field.

A polypeptide of the disclosure may also be produced by known conventional chemical synthesis. Methods for constructing the polypeptides of the disclosure by synthetic means are known to those skilled in the art. The synthetically-constructed polypeptide sequences, by virtue of sharing primary, secondary or tertiary structural and/or conformational characteristics with a native polypeptides may possess biological properties in common therewith, including biological activity.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Typically, the polypeptide of the disclosure is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Species homologues of procalins and polynucleotides encoding the polypeptides are also provided by the disclosure. As used herein, a “species homologue” is a polypeptide or polynucleotide with a different species of origin from that of a given polypeptide or polynucleotide, but with significant sequence similarity to the given polypeptide or polynucleotide. Species homologues may be isolated and identified by making suitable probes or primers from polynucleotides encoding the polypeptides provided herein and screening a suitable nucleic acid source from the desired species. Alternatively, homologues may be identified by screening a genome database containing sequences from one or more species utilizing a sequence (e.g., nucleic acid or amino acid sequence) of an procalin variant 1 and procalin variant 2 of the disclosure. Such genome databases are readily available for a number of species (e.g., on the world wide web (www) at tigr.org/tdb; genetics.wisc.edu; stanford.edu/.about.ball; hiv-web.lan1.gov; ncbi.nlm.nig.gov; ebi.ac.uk; and pasteur.fr/other/biology). The disclosure also encompasses allelic variants of procalins and nucleic acids encoding them that are naturally-occurring alternative forms of such polypeptides and polynucleotides in which differences in amino acid or nucleotide sequence are attributable to genetic polymorphism.

Intermediate Sequence Search (ISS) can be used to identify closely related as well as distant homologs by connecting two proteins through one or more intermediate sequences. ISS repetitively uses the results of the previous query as new search seeds. Saturated BLAST is a package that performs ISS. Starting with a protein sequence, Saturated BLAST runs a BLAST search and identifies representative sequences for the next generation of searches. The procedure is run until convergence or until some predefined criteria are met. Saturated BLAST is available on the world wide web (www) at: bioinformatics.burnham-inst.org/xblast (see also, Li et al. Bioinformatics 16(12): 1105, 2000).

Fragments of the procalins of the disclosure are encompassed by the disclosure and may be in linear form or cyclized using known methods (see, e.g., H. U. Saragovi, et al., Bio/Technology 10, 773 (1992); and R. S. McDowell, et al., J. Amer. Chem. Soc. 114:9245 (1992), both of which are incorporated by reference herein). Peptide fragments of procalins of the disclosure, and polynucleotides encoding such fragments include amino acid or nucleotide sequence lengths that are at least 25% (typically at least 50%, 60%, or 70%, and commonly at least 80%) of the length of an procalin or polynucleotide. Typically such sequences will have at least 60% sequence identity (more typically at least 70%-75%, 80°-85%, 90%-95%, at least 97.5%, or at least 99%, and most commonly at least 99.5%) with an procalin or polynucleotide when aligned so as to maximize overlap and identity while minimizing sequence gaps. Also included in the invention are polypeptides, peptide fragments, and polynucleotides encoding them, that contain or encode a segment comprising at least 6 to 10, typically at least 20, at least 30, or most commonly at least 40 contiguous amino acids. Such polypeptides and fragments may also contain a segment that shares at least 70% (at least 75%, 80%-85%, 90%-95%, at least 97.5%, or at least 99%, and commonly at least 99.5%) with any such segment of any of the MaSp family polypeptides, when aligned so as to maximize overlap and identity while minimizing sequence gaps. Visual inspection, mathematical calculation, or computer algorithms can determine the percent identity.

The disclosure also provides isolated polynucleotides and the polynucleotide sequences for the procalin 1 and procalin 2 polypeptides. In one aspect, the polynucleotides of the disclosure lack introns and thus procalin variant 1 and procalin variant 2 each possess only one enormous exon containing either 1,520 by (procalin 1) or 1,520 by (procalin 2) of coding sequence.

Also included are recombinant polypeptides and the polynucleotides encoding the polypeptides wherein the recombinant polypeptides are “chimeric polypeptides” or “fusion polypeptides” and comprise an procalin 1 or procalin 2 sequence as set forth in SEQ ID Nos: 2 or 4 operatively linked to a second polypeptide. The second polypeptide can be any polypeptide of interest having an activity or function independent of, or related to, the function of an Procalin variant 1 or Procalin variant 2 polypeptide. For example, the second polypeptide can be a domain of a related but distinct member of the procalin family of polypeptides. The term “operatively linked” is intended to indicate that the Procalin variant 1 or Procalin variant 2 sequence and the second polypeptide sequence are fused in-frame to each other. The second polypeptide can be fused to the N-terminus or C-terminus of an Procalin variant 1 or Procalin variant 2 sequence as set forth in FIGS. 1 and 2. For example, in one embodiment, the fusion polypeptide is a GST-Procalin variant 1 or Procalin variant 2 fusion polypeptide in which the Procalin variant 1 or Procalin variant 2 sequences are fused to the C-terminus of the GST sequences. Such fusion polypeptides can facilitate the purification of recombinant Procalin variant 1 or Procalin variant 2 polypeptides. In another embodiment, the fusion polypeptide comprises an Procalin variant 1 or Procalin variant 2 sequence comprising a heterologous signal sequence at its N-terminus. In certain host cells (e.g., mammalian host cells), expression and/or secretion of an Procalin can be increased through use of a heterologous signal sequence. As another example, an Procalin or fragment thereof may be fused to a hexa-histidine tag to facilitate purification of bacterially expressed protein, or to a hemagglutinin tag to facilitate purification of protein expressed in eukaryotic cells. Further, fusion polypeptides can comprise, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG peptide in the presence of certain divalent metal cations, as described in U.S. Pat. No. 5,011,912, hereby incorporated by reference. The 4E11 hybridoma cell line has been deposited with the ATCC under accession no. HB9259. Monoclonal antibodies that bind the FLAG peptide are available from Eastman Kodak Co., Scientific Imaging Systems Division, New Haven, Conn. In another embodiment, a procalin variant 1 or procalin variant 2 can be fused to an peptide or polypeptide useful as an adjuvant.

Encompassed by the disclosure are oligomers or fusion polypeptides that contain a procalin. Oligomers that can be used as fusion partners can be in the form of covalently linked or non-covalently-linked multimers, including dimers, trimers, or higher oligomers. In an alternative embodiment the disclosure is directed to oligomers comprising multiple polypeptides joined via covalent or non-covalent interactions between peptide moieties fused to the polypeptides. Such peptides can be peptide linkers (spacers), or peptides that have the property of promoting oligomerization. Leucine zippers and certain polypeptides derived from antibodies are among the peptides that can promote oligomerization of the polypeptides attached thereto, as described in more detail below.

Typically a linker will be a peptide linker moiety. The length of the linker moiety is chosen to optimize the biological activity of the polypeptide having an procalin variant 1 and procalin variant 2 sequence and can be determined empirically without undue experimentation. The linker moiety should be long enough and flexible enough to allow a procalin variant 1 and procalin variant 2 moiety to freely interact with a substrate or ligand. A linker moiety is a peptide between about one and 30 amino acid residues in length, typically between about two and 15 amino acid residues. Preferred linker moieties are—Gly-Gly-, GGGGS (SEQ ID NO:5), (GGGGS)ₙ, GKSSGSGSESKS (SEQ ID NO:6), GSTSGSGKSSEGKG (SEQ ID NO:7), GSTSGSGKSSEGSGSTKG (SEQ ID NO:8), GSTSGSGKPGSGEGSTKG (SEQ ID NO:9), or EGKSSGSGSESKEF (SEQ ID NO:10). Linking moieties are described, for example, in Huston, J. S., et al., PNAS 85:5879 (1988), Whitlow, M., et al., Protein Engineering 6:989 (1993), and Newton, D. L., et al., Biochemistry 35:545 (1996). Other suitable peptide linkers are those described in U.S. Pat. Nos. 4,751,180 and 4,935,233, which are hereby incorporated by reference. A DNA sequence encoding a desired peptide linker can be inserted between, and in the same reading frame as, DNA sequences of the disclosure, using any suitable conventional technique. For example, a chemically synthesized oligonucleotide encoding the linker can be ligated between the sequences. In particular embodiments, a fusion polypeptide comprises from two to four or more procalin variant 1 and/or procalin variant 2 polypeptides, separated by peptide linkers.

The procalins of the disclosure can also include a localization sequence to direct the polypeptide to particular cellular sites by fusion to appropriate organellar targeting signals or localized host proteins. A polynucleotide encoding a localization sequence, or signal sequence, can be ligated or fused at the 5′ terminus of a polynucleotide encoding a procalin such that the signal peptide is located at the amino terminal end of the resulting fusion polynucleotide or polypeptide. In eukaryotes, the signal peptide functions to transport a polypeptide across the endoplasmic reticulum. The secretory protein is then transported through the Golgi apparatus, into secretory vesicles and into the extracellular space or the external environment. Signal peptides include pre-pro peptides that contain a proteolytic enzyme recognition site.

The localization sequence can be a nuclear-, an endoplasmic reticulum-, a peroxisome-, or a mitochondrial-localization sequence, or a localized protein. Localization sequences can be targeting sequences that are described, for example, in “Protein Targeting”, chapter 35 of Stryer, L., Biochemistry (4th ed.). W.H. Freeman, 1995. Some important localization sequences include those targeting the nucleus (e.g., KKKRK (SEQ ID NO:11)), mitochondria (MLRTSSLFTRRVQPSLFRNILRLQST (SEQ ID NO:12), endoplasmic reticulum (KDEL (SEQ ID NO:13)), peroxisome (SKF), plasma membrane (CAAX (SEQ ID NO:14), CC, CXC, or CCXX (SEQ ID NO:15)), cytoplasmic side of plasma membrane (fusion to SNAP-25), or the Golgi apparatus (fusion to furin).

A chimeric or fusion polypeptide of the disclosure can be produced by standard recombinant molecular biology techniques. In one embodiment, polynucleotide fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Examples of polynucleotides encoding all or portions of the procalins are set forth in SEQ ID NO:1 or 3. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide).

The disclosure further includes polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the disclosure in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase.

Additional variants within the scope of the disclosure include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Typically, such alteration, substitution, replacement, insertion or deletion retains the desired activity of the polypeptide.

The disclosure also provides polynucleotides encoding Procalins. The term “polynucleotide” refers to a polymeric form of nucleotides of at least 10 bases in length. The nucleotides can be ribonucleotides, deoxyribonucleotides, or modified forms of either type of nucleotide. The term includes single and double stranded forms of DNA or RNA. DNA includes, for example, cDNA, genomic DNA, chemically synthesized DNA, DNA amplified by PCR, and combinations thereof. The polynucleotides of the disclosure include full-length genes and cDNA molecules as well as a combination of fragments thereof. The polynucleotides of the disclosure are derived from a Triatoma sp.

A polynucleotide of the disclosure will generally contain phosphodiester bonds, although in some cases, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made;

alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Other analogs include peptide nucleic acids (PNA) which are peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tᵣ, for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

As described above, the nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant polynucleotide molecule, which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

A procalin variant 1 or procalin variant 2 polynucleotide of the disclosure (1) encodes a polypeptide comprising a sequence as set forth in SEQ ID Nos: 2 or 4; (2) comprises a sequence as set forth in SEQ ID Nos:1 or 3; (3) comprises sequences complementary to a sequence as set forth in SEQ ID Nos:1 or 3; (4) fragments of SEQ ID Nos:1 or 3 or their complements that specifically hybridize to the polynucleotide consisting of the sequence as set forth in (2) or (3) above under moderate to highly stringent conditions; and (5) polynucleotides of (1), (2), (3), or (4) wherein T can also be U (e.g., RNA sequences). Also encompassed by the disclosure are homologs of an procalin variant 1 and procalin variant 2 polynucleotide of the disclosure. These polynucleotides can be identified in several ways, including isolation of genomic or cDNA molecules from a suitable source, or computer searches of available sequence databases. Oligonucleotides or polynucleotides corresponding to the amino acid sequences described herein can be used as probes or primers for the isolation of polynucleotide homologs or as query sequences for database searches. Degenerate oligonucleotide sequences can be obtained by “back-translation” from the amino acid sequences of the disclosure. The polymerase chain reaction (PCR) procedure can be employed to isolate and amplify a DNA sequence encoding an procalin polypeptide. Oligonucleotides that define the desired termini of a target DNA molecule are employed as 5′ and 3′ primers. Accordingly, fragments of the polynucleotides of the disclosure are useful as probes and primers to identify or amplify related sequence or obtain full-length sequences of a procalin variant 1 and procalin variant 2 of the disclosure. The oligonucleotides can additionally contain recognition sites for restriction endonucleases, to facilitate insertion of the amplified combination of DNA fragments into an expression vector. PCR techniques are known in the art (see, e.g., PCR Protocols: A Guide to Methods and Applications, Innis et. al., eds., Academic Press, Inc. (1990)).

Among the uses of the disclosed procalin variant 1 and procalin variant 2 polynucleotides, and combinations of fragments thereof, is the use of fragments as probes or primers. Such fragments generally comprise at least about 17 contiguous nucleotides of a DNA sequence. In other embodiments, a DNA fragment comprises at least 30, or at least 60 contiguous nucleotides of a DNA sequence. In one embodiment, a probe or primer comprises at least 15-30 nucleotide bases containing nucleotides 76-78, 85-87, 442-444, or 502-504 of SEQ ID NO:1 or 3. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In another embodiment, the probes are useful as diagnostic to identify, for example, the presence of a particular allergen. In certain embodiments, degenerate primers can be used as probes for non-human genetic libraries. Such libraries would include but are not limited to cDNA libraries, genomic libraries, and even electronic EST (express sequence tag) or DNA libraries. Homologous sequences identified by this method would then be used as probes to identify non-human homologues of the procalin variant 1 and procalin variant 2 sequences identified herein.

The disclosure also includes polynucleotides and oligonucleotides that hybridize under reduced stringency conditions, typically moderately stringent conditions, and commonly highly stringent conditions, to procalin variant 1 or procalin variant 2 polynucleotides described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, F. M. Ausubel et al., eds., John Wiley & Sons, Inc., sections 2.10 and 6.3-6.4, incorporated herein by reference), and can be readily determined by those having ordinary skill in the art based on, for example, the length and/or base composition of the polynucleotide. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target polynucleotide of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tₘ) of the hybrid, where Tₘ is determined according to the following equations. For hybrids less than 18 base pairs in length, Tᵣ, (° C.)=2(# of A+T bases)+4(# of G+C bases). For hybrids above 18 base pairs in length, Tᵣ, (° C.)=81.5+16.6 (log₁₀ [Na⁺])+0.41 (% G+C)−(600/N), where N is the number of bases in the hybrid, and [Na⁺] is the concentration of sodium ions in the hybridization buffer ([Na⁺] for 1×SSC=0.165M). Preferably, each such hybridizing nucleic acid has a length that is at least 25% (more preferably at least 50%, 60%, or 70%, and most preferably at least 80%) of the length of a polynucleotide of the disclosure to which it hybridizes, and has at least 60% sequence identity (more preferably at least 70%, 75%, 80%, 85%, 90%, 95%, 97.5%, or at least 99%, and most preferably at least 99.5%) with a polynucleotide of the disclosure to which it hybridizes.

“Conservatively modified variants” applies to both polypeptide and polynucleotide. With respect to particular polynucleotide, conservatively modified variants refer to codons in the polynucleotide which encode identical or essentially identical amino acids. Because of the degeneracy of the genetic code, a large number of functionally identical polynucleotides encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such variations are “silent variations,” which are one species of conservatively modified variations. Every polynucleotide sequence herein that encodes a polypeptide also describes every possible silent variation of the nucleic acid. One of skill will recognize that each codon in a polynucleotide (except AUG, which is ordinarily the only codon for methionine) can be modified to yield a functionally identical molecule. Accordingly, each silent variation of a nucleic acid that encodes a polypeptide is implicit in each described sequence.

The disclosure also provides methodology for analysis of polynucleotides of the disclosure on “DNA chips” as described in Hacia et al., Nature Genetics, 14:441-447 (1996). For example, high-density arrays of oligonucleotides consisting of a sequence as set forth in SEQ ID Nos:1 or 3, or a variant or mutant thereof are applied and immobilized to the chip and can be used to detect sequence variations in a population. Polynucleotides in a test sample are hybridized to the immobilized oligonucleotides. The hybridization profile of the target polynucleotide to the immobilized probe is quantitated and compared to a reference profile. The resulting genetic information can be used in molecular identification. The density of oligonucleotides on DNA chips can be modified as needed.

The disclosure also provides genes corresponding to the polynucleotides disclosed herein. “Corresponding genes” are the regions of the genome that are transcribed to produce the mRNAs from which cDNA molecules are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials.

Expression, isolation, and purification of the polypeptides and fragments of the disclosure can be accomplished by any suitable technique including, but not limited to, the following methods and those described elsewhere herein.

The isolated polynucleotides of the disclosure may be operably linked to an expression control sequence such as the pMT2 or pED expression vectors disclosed in Kaufman et al., Nucleic Acids Res. 19:4485 (1991); and Pouwels et al. Cloning Vectors: A Laboratory Manual, Elsevier, N.Y., (1985, and Supplements), in order to produce a polypeptide of the disclosure recombinantly. Many suitable expression control sequences are known in the art. General methods of expressing recombinant polypeptides are also known and are exemplified in R. Kaufman, Methods in Enzymology 185:537 (1990). As defined herein “operably linked” means that an isolated polynucleotide of the disclosure and an expression control sequence are situated within a vector or cell in such a way that the polypeptide encoded by the polynucleotide is expressed by a host cell which has been transformed (transfected) with the vector or polynucleotide operably linked to the control sequence.

For example, expression of a procalin polypeptide can be performed in E. coli by inserting the polyncleotide encoding procalin variant 1 and/or procalin variant 2 into plasmid vectors pFP202 and pFP204, which can be derived from the well-known vector pET11a. In these vectors, the procalin-coding gene is inserted in such a manner as to be operably linked to a promoter derived from bacteriophage T7. This promoter is joined with sequences derived from the lac operator of E. coli, which confers regulation by lactose or analogs (IPTG). The E. coli host strain BL21(DE3) contains a lambda prophage which carries a gene encoding bacteriophage T7 RNA polymerase. This gene is controlled by a promoter which is also regulated by lactose or analogs. In addition to the phage T7 promoter, the vectors pFP202 and pFP204 provide sequences which encode a C-terminal tail containing six consecutive histidine residues appended to the procalin-coding sequences. This tail provides a means of affinity purification of the protein under denaturing conditions through its adsorption to resins bearing immobilized Ni ions.

In addition, a sequence encoding an appropriate signal peptide (native or heterologous) can be incorporated into expression vectors. The choice of signal peptide or leader can depend on factors such as the type of host cells in which the recombinant polypeptide is to be produced. Examples of heterologous signal peptides that are functional in mammalian host cells include the signal sequence for interleukin (IL)-7 (see, U.S. Pat. No. 4,965,195); the signal sequence for IL-2 receptor (see, Cosman et al., Nature 312:768, 1984); the IL4 receptor signal peptide (see, EP 367,566); the type I IL-1 receptor signal peptide (see, U.S. Pat. No. 4,968,607); and the type II IL-1 receptor signal peptide (see, EP 460,846). A signal peptide that is functional in the intended host cells promotes extracellular secretion of the polypeptide. The signal peptide is cleaved from the polypeptide upon secretion of a polypeptide from the cell. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine or Lipofectamine-Plus lipid reagent (Gibco/BRL), can be used to transfect cells (Felgner et al., Proc. Natl. Acad. Sci. USA 84:7413, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487, 1990, describes several selection schemes, such as dihydrofolate reductase (DHFR) resistance. A suitable strain for DHFR selection can be CHO strain DX-B11, which is deficient in DHFR (Urlaub et al., Proc. Natl. Acad. Sci. USA 77:4216, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector are selected on the basis of resistance to these compounds.

Alternatively, gene products can be obtained via homologous recombination, or “gene targeting” techniques. Such techniques employ the introduction of exogenous transcription control elements (such as the CMV promoter or the like) in a particular predetermined site on the genome, to induce expression of an endogenous procalin variant 1 and procalin variant 2 of the disclosure. The location of integration into a host chromosome or genome can be easily determined by one of skill in the art, given the known location and sequence of the gene. In one embodiment, the disclosure also contemplates the introduction of exogenous transcriptional control elements in conjunction with an amplifiable gene, to produce increased amounts of the gene product. The practice of homologous recombination or gene targeting is explained by Chappel in U.S. Pat. No. 5,272,071 (see also Schimke, et al. “Amplification of Genes in Somatic Mammalian cells,” Methods in Enzymology 151:85 (1987), and by Capecchi, et al., “The New Mouse Genetics Altering the Genome by Gene Targeting,” TIG 5:70 (1989)).

Suitable host cells for expression of the polypeptide include eukaryotic, insect and prokaryotic cells. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) (see, McMahan et al. EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it may be possible to produce the polypeptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous polypeptides. Potentially suitable bacterial strains include, for example, Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments may be accomplished using known chemical or enzymatic methods. The polypeptide may also be produced by operably linking a polynucleotide of the disclosure to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac.RTM. kit), as well as methods described in Summers and Smith, Texas Agricultural Experiment Station Bulletin No. 1555 (1987), and Luckow and Summers, Bio/Technology 6:47 (1988), incorporated herein by reference. Cell-free translation systems could also be employed to produce polypeptides using RNAs derived from nucleic acid constructs disclosed herein. A host cell that comprises an isolated polynucleotide of the disclosure, typically operably linked to at least one expression control sequence, is a “recombinant host cell”.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a mRNA having an Procalin variant 1 and Procalin variant 2 polynucleotide sequence. Absolute complementarity, although preferred, is not required. Oligonucleotides that are complementary to the 5′ end of the message, e.g., the 5′ untranslated sequence up to, and including, the AUG initiation codon, should work most efficiently at inhibiting translation. Antisense nucleic acids are preferably oligonucleotides ranging from 6 to about 50 nucleotides in length. The oligonucleotides can be DNA, RNA, chimeric mixtures, derivatives or modified versions thereof, single-stranded or double-stranded. The oligonucleotide can be modified at the base moiety, sugar moiety, or phosphate backbone, for example, to improve stability of the molecule, hybridization, and the like. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553, 1989; Lemaitre et al., Proc. Natl. Acad. Sci. 84:648, 1987; PCT Publication No. WO88/09810), or hybridization-triggered cleavage agents or intercalating agents (see, e.g., Zon, Pharm. Res. 5:539, 1988). The antisense molecules are delivered to cells, which express a transcript having an Procalin variant 1 and Procalin variant 2 polynucleotide sequence in vivo by, for example, direct injection into the tissue or cell derivation site, or modified antisense molecules, designed to target the desired cells (e.g., antisense linked to peptides or antibodies that specifically bind receptors or antigens expressed on the target cell surface) can be administered systemically. Preferred approach utilizes a recombinant DNA construct in which the antisense oligonucleotide is placed under the control of a strong pol III or pol II promoter.

Ribozyme molecules designed to catalytically cleave mRNA transcripts having an Procalin variant 1 and Procalin variant 2 polynucleotide sequence prevent translation of Procalin variant 1 and Procalin variant 2 mRNA (see, e.g., PCT International Publication WO90/11364; U.S. Pat. No. 5,824,519). Ribozymes are RNA molecules possessing the ability to specifically cleave other single-stranded RNA. Because ribozymes are sequence-specific, only mRNAs with particular sequences are inactivated. There are two basic types of ribozymes namely, tetrahymena-type (Hasselhoff, Nature, 334:585, 1988) and “hammerhead”-type. Tetrahymena-type ribozymes recognize sequences, which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes. As in the antisense approach, ribozymes can be composed of modified oligonucleotides and delivered using a DNA construct “encoding” the ribozyme under the control of a strong constitutive pol III or pol II promoter.

Alternatively, endogenous Procalin variant 1 and Procalin variant 2 expression can be reduced by targeting DNA sequences complementary to a regulatory region of the target gene (e.g., the target gene promoter and/or enhancers) to form triple helical structures that prevent transcription of the target gene (see generally, Helene, Anticancer Drug Des., 6(6), 569, 1991; Helene, et al., Ann. N.Y. Acad. Sci., 660:27, 1992; and Maher, Bioassays 14(12), 807, 1992).

Antisense, ribozyme, and triple helix molecules of the disclosure may be prepared by any method known in the art for the synthesis of DNA and RNA molecules and include techniques for chemically synthesizing oligodeoxyribonucleotides and oligoribonucleotides such as, for example, solid phase phosphoramidite chemical synthesis using an automated DNA synthesizer available from Biosearch, Applied Biosystems. Phosphorothioate oligonucleotides may be synthesized by the method of Stein et al., Nucl. Acids Res. 16:3209, 1988. Methylphosphonate oligonucleotides can be prepared by use of controlled pore glass polymer supports (Sarin et al., Proc. Natl. Acad. Sci. U.S.A. 85:7448, 1988). Alternatively, RNA molecules may be generated by in vitro and in vivo transcription of DNA sequences encoding the antisense RNA molecule.

In another embodiment, antibodies that are immunoreactive with the polypeptides of the disclosure are provided herein. The procalins, fragments, variants, fusion polypeptides, and the like, as set forth above, can be employed as “immunogens” in producing antibodies immunoreactive therewith. Such antibodies specifically bind to the polypeptides via the antigen-binding sites of the antibody. Specifically binding antibodies are those that will specifically recognize and bind with procalin variant 1 and procalin variant 2 family polypeptides, homologues, and variants, but not with other molecules. In one embodiment, the antibodies are specific for polypeptides having an procalin variant 1 or procalin variant 2 amino acid sequence of the disclosure and do not cross-react with other polypeptides or procalin polypeptide family members.

More specifically, the polypeptides, fragment, variants, fusion polypeptides, and the like contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding. Epitopes can be identified by any of the methods known in the art. Additionally, epitopes from the polypeptides of the disclosure can be used as research reagents, in assays, and to purify specific binding antibodies from substances such as polyclonal sera or supernatants from cultured hybridomas. Such epitopes or variants thereof can be produced using techniques known in the art such as solid-phase synthesis, chemical or enzymatic cleavage of a polypeptide, or using recombinant DNA technology.

Both polyclonal and monoclonal antibodies to the polypeptides of the disclosure can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kosbor et al., Immunology Today 4:72, 1983; Cole et al., Proc. Natl. Acad. Sci. USA 80:2026, 1983); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the disclosure are also contemplated herein. Such hybridomas can be produced and identified by conventional techniques. For the production of antibodies, various host animals may be immunized by injection with an procalin, fragment, variant, or mutants thereof. Such host animals may include, but are not limited to, rabbits, mice, and rats, to name a few. Various adjutants may be used to increase the immunological response. Depending on the host species, such adjutants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies may be of any immunoglobulin class including IgG, IgM, IgE, IgA, IgD, and any subclass thereof.

Antibody fragments, which recognize specific epitopes, may be generated by known techniques. For example, such fragments include but are not limited to: the F(ab′)₂ fragments which can be produced by pepsin digestion of the antibody molecule and the Fab fragments which can be generated by reducing the disulfide bridges of the (ab′)₂ fragments. Alternatively, Fab expression libraries may be constructed (Huse et al., Science, 246:1275, 1989) to allow rapid and easy identification of monoclonal Fab fragments with the desired specificity. Techniques described for the production of single chain antibodies (U.S. Pat. No. 4,946,778; Bird, Science 242:423, 1988; Huston et al., Proc. Natl. Acad. Sci. USA 85:5879, 1988; and Ward et al., Nature 334:544, 1989) can also be adapted to produce single chain antibodies against polypeptides containing Procalin variant 1 and Procalin variant 2 amino acid sequences.

The antibodies of the disclosure can also be used in assays to detect the presence of the polypeptides or fragments of the disclosure, either in vitro or in vivo. The antibodies also can be employed in purifying polypeptides or fragments of the disclosure by immunoaffinity chromatography.

The disclosure provides methods for identifying agents that modulate procalin variant 1 and procalin variant 2 activity or expression. Such methods included contacting a sample containing a procalin or polynucleotide with a test agent under conditions that allow for the test agent and the polypeptide or polynucleotide to interact and measuring the expression or activity of a procalin in the presence or absence of the test agent.

This disclosure provides vaccine antigens useful for providing protective immunity. A vaccine “induces” an immune response when the antigen or antigens present in the vaccine cause the vaccinated subject to mount an immune response to that antigen or antigens. The vaccinated subject will generate an immune response, as evidenced by activation of the immune system, which includes the production of vaccine antigen-specific T cells, vaccine antigen-specific B cells, vaccine antigen-specific antibodies, and cytokines. The resulting immune response may be measured by several methods including ELISPOT, ELISA, chromium release assays, intracellular cytokine staining, FACS analysis, and MHC tetramer staining (to identify peptide-specific cells). A skilled artisan may also use these methods to measure a primary immune response or a secondary immune response. Such vaccines can be used to increase the immune tolerance to a particular antigen by repeated low-dose administration.

Certain vaccine adjuvants are particularly suited to the stimulation of either Th1 or Th2-type cytokine responses. Traditionally, the best indicators of the Th1:Th2 balance of the immune response after a vaccination or infection includes direct measurement of the production of Th1 or Th2 cytokines by T lymphocytes in vitro after restimulation with antigen, and/or the measurement of the IgG1:IgG2a ratio of antigen specific antibody responses. Thus, a Th1-type adjuvant is one which stimulates isolated T-cell populations to produce high levels of Th1-type cytokines when re-stimulated with antigen in vitro, and induces antigen specific immunoglobulin responses associated with Th1-type isotype.

The disclosure provides methods of stimulating an immune response in a subject against a Triatoma sp. by administering to the subject an immunogenic amount of a procalin antigen(s), wherein the administration results in the production of an immune response. The procalin 1 or 2 antigen(s) preparation can be administered in combination with an immunostimulant adjuvant.

The disclosure further relates to antibodies for the prevention and/or treatment of a Triatoma sp. reaction or infection. In one embodiment, an antibody is raised against a procalin antigen of the disclosure. Such antibodies are produced by administering an antigenic composition comprising a vector expression, or a purified preparation of, a procalin antigen as a vaccine.

The antibodies according to the disclosure will be administered in one or more dosages, and the amount needed will depend on during which phase of the reaction the therapy is given as well as on other factors. In order to produce antibodies, the antigenic composition according to the disclosure will be administered to a subject in order to induce the production of the above described antibodies characteristic for a procalin of the disclosure. The antibodies can be monoclonal antibodies. Once designed, such novel antibodies may be produced by conventional techniques and used in therapy. In general, a monoclonal antibody to an epitope of an antigen can be prepared by using a technique which provides for the production of antibody molecules from continuous cell lines in culture and methods of preparing antibodies are well known to the skilled in this field (see e.g. Coligan (1991) Current Protocols in Immunology, Wiley/Greene, N.Y.; Harlow and Lane (1989) Antibodies: A Laboratory Manual, Cold Spring Harbor Press, NY; and Goding (1986) Monoclonal Antibodies: Principles and Practice (2.sup.nd ed) Academic Press, New York, N.Y.). For therapeutic purposes, there may be an interest in using human antibodies.

For therapeutic purposes, the antibody is typically formulated with conventional pharmaceutically or pharmacologically acceptable vehicles for administration, conveniently by injection. Vehicles include deionized water, saline, phosphate-buffered saline, Ringer's solution, dextrose solution, Hank's solution, etc. Other additives may include additives to provide isotonicity, buffers, preservatives, and the like. The antibody may be administered parenterally, typically intravenously or intramuscularly, as a bolus, intermittently or in a continuous regimen.

Methods for ameliorating a reaction in a subject by administering to the subject a procalin antigen(s) or a vector comprising a procalin antigen, in a pharmaceutically acceptable carrier, are also provided.

The immunogenic compositions and vaccines obtained using the methods of the disclosure can be formulated as pharmaceutical compositions for administration in any suitable manner. One route of administration is oral. Other routes of administration include rectal, intrathecal, buccal (e.g., sublingual) inhalation, intranasal, intradermal, intramuscular, and transdermal and the like (see e.g. U.S. Pat. No. 6,126,938). Although more than one route can be used to administer a particular composition, a particular route can often provide a more immediate and more effective reaction than another route.

The immunoprotective compositions to be administered are provided in a pharmaceutically acceptable solution such as an aqueous solution, often a saline or buffered solution, or they can be provided in powder form. There is a wide variety of suitable formulations of pharmaceutical compositions of the disclosure. See, e.g., Lieberman, Pharmaceutical Dosage Forms, Marcel Dekker, Vols. 1-3 (1998); Remington's Pharmaceutical Science, 17th ed., Mack Publishing Company, Easton, Pa. (1985) and similar publications. The compositions may also include an adjuvant. Examples of known suitable adjuvants include alum, aluminum phosphate, aluminum hydroxide, and MF59 (4.3% w/v squalene, 0.5% w/v Tween 80, 0.5% w/v Span 85)—these are the only ones currently licensed for use in humans. For experimental animals, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, or Bacille Calmette-Guerin (BCG). The effectiveness of an adjuvant may be determined by measuring the amount of antibodies directed against the immunogenic antigen.

The concentration of immunogenic antigens of the disclosure in the pharmaceutical formulations can vary widely, e.g., from less than about 0.1%, usually at or at least about 2% to as much as 20% to 50% or more by weight, and will be selected primarily by fluid volumes, viscosities, and the like, in accordance with the particular mode of administration selected.

Formulations suitable for oral administration can comprise (a) liquid solutions, such as buffered water, saline or PEG 400; (b) capsules, sachets or tablets, each containing a predetermined amount of the active ingredient, as lyophilized powder, liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

The dose administered to a subject, in the context of the disclosure should be sufficient to affect a beneficial therapeutic and/or prophylactic response in the subject over time. The dose will be determined by the efficacy of the particular attenuated vaccine employed and the condition of the subject, as well as the body weight or vascular surface area of the subject to be treated. The size of the dose also will be determined by the existence, nature, and extent of any adverse side-effects that accompany the administration of a particular vaccine in a particular subject.

In determining the effective amount of the vaccine to be administered in the treatment or prophylaxis of an infection or other condition, the physician evaluates vaccine toxicities, progression of the disease, and the production of anti-vaccine vector antibodies, if any.

The compositions are administered to a subject that is at risk of being exposed to a Triatoma sp. or to prevent or at least partially arrest the development of a reaction and its complications. An amount adequate to accomplish this is defined as a “therapeutically effective dose.” Amounts effective for therapeutic use will depend on, e.g., the antigen composition, the manner of administration, the weight and general state of health of the subject, and the judgment of the prescribing physician. Single or multiple doses of the antigen compositions may be administered depending on the dosage and frequency required and tolerated by the subject, and route of administration. In addition, a booster may be administered in the same or different formulation.

The following examples are meant to illustrate the invention but should not be construed as limiting it in any way.

### EXAMPLES

Triatoma protracta were collected in San Diego County, Calif. T. protracta salivary glands were dissected, and total RNA was extracted from the salivary glands using Trizol Reagent (Invitrogen) to lyse the cells, followed by addition of chloroform, and then precipitated with isopropyl alcohol. The RNA was reverse transcribed using Superscript III Reverse Transcriptase (Invitrogen) and Oligo dT. First-strand cDNA was amplified using PCR with Accuprime High Fidelity Taq DNA polymerase (Invitrogen):

forward primer 5′ - TCGCCAACATGAAGACG - 3′
reverse primer 5′ - ATTTAAAATAATCACTTCAAAC - 3′

Both primer sequences were derived from previously published procalin sequence (Paddock, 2001). The amplified product was gel-purified and subcloned into the pGEM-T vector system (Promega). The forward and reverse strands of the recombinant DNA were sequenced, resulting in two closely related sequences, possibly representing subspecies variants (differences underlined and in bold) (SEQ ID NO:1 (top), SEQ ID NO:3 (bottom):

5′ - ATGAAGACGTTCATTGTAATCACATTTATTGGAATCCTGTCCTACGCATATG - 3′
5′ - ATGAAGACGTTCATTGTAATCACATTTATTGGAATCCTGTCCTACGCATATG - 3′
5′ - CTGCAAACGAATGTGAAAATCCAGAACCTATGCAAGGTTTTAGTGCCTCAC - 3′
5′ - CTGCAAACGAATGTGAAAATCCAAAACCTATGGAAGGTTTTAGTGCCTCAC - 3′
5′ - AATTTTACCAAGGAAAGTGGTATGTGACTCATGAAACATCCGCATCAACTC - 3′
5′ - AATTTTACCAAGGAAAGTGGTATGTGACTCATGAAACATCCGCATCAACTC - 3′
5′ - TATCCGAATGTAACATTCTTACTACTAGCAATGATAATGGCAAATTCACTGT - 3′
5′ - TATCCGAATGTAACATTCTTACTACTAGCAATGATAATGGCAAATTCACTGT - 3′
5′ - TAAACATAAATATACCAAAGATGGAAAAGTGGGTGAACTCATTTGTGAGGG - 3′
5′ - TAAACATAAATATACCAAAGATGGAAAAGTGGGTGAACTCATTTGTGAGGG - 3′
5′ - TCAAGCAAGCGCAAATAATAAATTTACTTACGACTGCAAATTTGACGGAGA - 3′
5′ - TCAAGCAAGCGCAAATAATAAATTTACTTACGACTGCAAATTTGACGGAGA - 3′
5′ - GACTATGGAGCAAGTAACTCGAACAGCAATGGATACAGACTATAATGACTA - 3′
5′ - GACTATGGAGCAAGTAACTCGAACAGCAATGGATACAGACTATAATGACTA - 3′
5′ - CGCTCTGTACTATTTATGTACAACTTATAAAAATGGTCCTAATGCTGGCAAG - 3′
5′ - CGCTCTGTACTATTTATGTACAACTTATAAAAATGGTCCTAATGCTGGCAAG - 3′
5′ - AAAGAAGGGCATTATATTCTTTCAAGAAGACAACCAAATACAGAAATTCCT - 3′
5′ - AAAGAAGGGCATTATATTCTTTCAAGAAGAAAACCAAATACAGAAATTCCT - 3′
5′ - GATGCGCTGAAAACTAAGACAAAGGATTTGAATTTGAAATTATGTGGTTAA - 3′
5′ - GATGCGCTGAAAACTAAGACAAAGGATTTGAATTTGAAAATATGTGGTTAA - 3′

The translated amino acid sequence alignment is shown below (SEQ ID NO:2 and 4, respectively):

5′-MK↓TFIVITFIGILSYAYAANECENPEPMQGFSASQFYQGKWYVTHETSASTLSE-3′
5′-MK↓TFIVITFIGILSYAYAANECENPKPMEGFSASQFYQGKWYVTHETSASTLSE-3′
5′-CNILTTSNDNGKFTVKHKYTKDGKVGELICEGQASANNKFTYDCKEDGETMEQV-3′
5′-CNILTTSNDNGKFTVKHKYTKDGKVGELICEGQASANNKFTYDCKEDGETMEQV-3′
5′-TRTAMDTDYNDYALYYLCTTYKNGPNAGKKEGHYILSRRQPNTEIPDALKTKTK-3′
5′-TRTAMDTDYNDYALYYLCITYKNGPNAGKKEGHYILSRRKPNTEIPDALKTKIK-3′
5′-DLNLKLCG-3′
5′-DLNLKICG-3′

Examination of the nucleotide and amino acid sequences shows that there are three additional nucleotides when compared to a published sequence (Paddock, 2001), resulting in an additional alanine and changes an aspartic acid to asparagines (sequences underlined and in italics). To confirm the findings, genomic DNA was purified from T. protracta tissues lysed with proteinase K. Forward primer 5′-CCTGTCCTACGCATATGC-3′ and reverse primer 5′-GCATAGGTTCTGGATTTTCAC-3′ were used in a PCR reaction to specifically isolate the region of interest. Similarly, the amplified product was gel-purified, subcloned into pGEM-T vector, and sequenced. The DNA sequence of the specific region amplified was identical to the procalin sequence amplified from RNA, validating the original cloned sequence. The present sequences therefore supersede the previously published sequence from Paddock et al. and identify variants among the wild Triatoma population that may affect clinical allergic responses and diagnostic test accuracy.

Using the BaculoDirect Baculovirus Expression System (Invitrogen), the procalin protein was produced for use in further studies of allergenicity. The procalin gene was removed from the pGEM-T vector at restriction sites and recombined into the pEntr3C vector. The pEntr3C vector contains an att-specific attachment site, where recombination with the att site on the BaculoDirect Linear DNA occurs; thus, a direct transfer of the gene into the baculovirus genome. The resulting recombinant DNA was transfected into SF9 insect cells. After several rounds of ganciclovir selection, a high titer stock of recombinant virus was made and can subsequently be used to transfect insect cells to produce recombinant procalin protein secreted into the supernatant. Since the BaculoDirect Linear DNA carries a C-terminal V5-His tag, the tag was fused to the c-terminal end of the product recombinant procalin protein, which was then used for the purification of the protein. A western blot analysis of the crude procalin protein collected from the supernatant of transfected SF9 cells is shown in FIG. 3. The upper band represents the 20 kD procalin protein. The lower band is suspected to be the result of trypsin digestion at the site indicated by an arrow on the previous amino acid sequence shown above.

To determine the allergic reactions of subject to triatoma insects the following experiments were performed. 3.38 mg of recombinant procalin in 1.5 ml of 10% PBS and 10 mM HEPES was adjusted to pH 7-9 with 1N NaOH. 300 μl of a 10 mM aqueous solution of biotin was added to 1.5 ml of procalin in a microcentrifuge tube and mixed in an end-to-end rotator at room temperature for 2 hours. (Biotin to protein ratio ˜10:1). The biotinylated procalin solution was transferred into a dialysis tube (MW cutoff of 1 kDa) and dialyzed against PBS to remove and inactivate unbound biotin. Following dialysis, the protein concentration was measured and adjusted to ˜1 mg/ml by ultrafiltation. 50 μg of the biotinylated procalin was loaded onto Streptavidin solid phases (Phadia ImmunoCap® # Ro212) and incubated at 37° C. for 1 hour.

Allergic reactions including anaphylaxis to the bite of triatomine insects has been well described in the literature. Diagnostic tests for confirmation of triatoma allergy have been limited to skin tests using extracts prepared from whole body extracts of Triatoma protracta (TP). There is no standardized, commercially available in vitro test for confirming triatoma allergy. Accordingly, the disclosure provides a test based upon procalin polypeptides.

cDNA encoding procalin was produced and used to generate large amounts of recombinant protein antigen which was then biotinylated and coupled to streptavidin ImmunoCAPs. The resulting procalin-bound ImmunoCAPs were then used to test the serum of 9 subjects with histories of triatoma allergy and two non-allergic controls using the Phadia ImmunoCAP 1000 system. Results were compared to ImmunoCAPs produced on a research basis for us by Phadia® using whole body extracts of TP.

The two non-allergic subjects revealed levels of Triatoma-specific IgE of <0.1 KUA/L with both procalin ImmunoCAPs and whole body triatoma extract ImmunoCAPs (wbe). Triatoma-specific IgE was detected in 7/9 patients with a history of triatoma allergy. 3 subjects were positive to both wbe and procalin, 2 were positive to wbe but not procalin, 2 were positive to procalin but not wbe and 2 were negative to both wbe and procalin

These results indicate that subjects allergic to triatoma bites react on in vitro testing for IgE antibodies to wbe and to the salivary antigen procalin. The lack of complete convergence between these two antigen sources suggests that patients with triatoma allergy may react to triatoma allergens other than procalin.

TABLE 1
Specific IgE levels to whole body extract
and procalin in individuals with a history compatible with an
allergic reaction to triatoma.

| | Whole body extract | |
|---|---|---|
| Subject | (KU_{A}/L) | Procalin (KU_{A}/L) |
| BL | 3.82 | 5.2 |
| MH | <0.1 | 0.37 |
| LT | 0.4 | <0.1 |
| CC | 0.58 | <0.1 |
| DA | 0.24 | 0.94 |
| FO | <0.1 | 44.6 |
| MW | 0.2 | 5.65 |
| KS | <0.1 | <0.1 |
| ED | <0.1 | <0.1 |

Controls

| | | |
|---|---|---|
| DR | <0.1 | <0.1 |
| JN | <0.1 | <0.1 |

Although certain embodiments have been demonstrated, the foregoing examples are intended to illustrate but not limit the following claims.

What is claimed is:

1. An isolated procalin 1 polypeptide, comprising:
(i) a polypeptide encoded by SEQ ID NO:1,
(ii) a polypeptide comprising SEQ ID NO:2,
(iii) a polypeptide having at least 80%, 90%, 95%, 98%, 99% identity to SEQ ID NO:2 and having anticoagulant effect, vasodilatoryeffect or a combination thereof,
(iv) a polypeptide comprising SEQ ID NO:2 and having at least 1-30 (e.g., 1-10, 5-15, 10-20, or 15-30) conservative amino acid substitutions and having anticoagulant effect, vasodilatory effect or a combination thereof,
(v) a polypeptide having at least 80%, 90%, 95%, 98%, 99% identity to SEQ ID NO:2 and having a Glu at position 26, a Gln at position 29, a Gln at position 148, a Leu at position 168, and any combination thereof and having anticoagulant effect, vasodilatoryeffect or a combination thereof,
(vi) a polypeptide comprising SEQ ID NO:2 and having at least 1-30 (e.g., 1-10, 5-15, 10-20, or 15-30) conservative amino acid substitutions, and having a Glu at position 26, a Gln at position 29, a Gln at position 148, a Leu at position 168, and any combination thereof and having anticoagulant effect, vasodilatoryeffect or a combination thereof, or
(vii) a polypeptide comprising a fragment of any of the foregoing wherein an antibody raised against the fragment specifically binds to a procalin 1 polypeptide.

2. A procalin 2 polypeptide, comprising:
(i) a polypeptide encoded by SEQ ID NO:3,
(ii) a polypeptide comprising SEQ ID NO:4,
(iii) a polypeptide having at least 80%, 90%, 95%, 98%, 99% identity to SEQ ID NO:4 and having anticoagulant effect, vasodilatoryeffect or a combination thereof,
(iv) a polypeptide comprising SEQ ID NO:4 and having at least 1-30 (e.g., 1-10, 5-15, 10-20, or 15-30) conservative amino acid substitutions and having anticoagulant effect, vasodilatory effect or a combination thereof,
(v) a polypeptide having at least 80%, 90%, 95%, 98%, 99% identity to SEQ ID NO:4 and having a Lys at position 26, a Glu at position 29, a Lys at position 148, a Iso at position 168, and any combination thereof and having anticoagulant effect, vasodilatoryeffect or a combination thereof,
(vi) a polypeptide comprising SEQ ID NO:4 and having at least 1-30 (e.g., 1-10, 5-15, 10-20, or 15-30) conservative amino acid substitutions, and having a Lys at position 26, a Glu at position 29, a Lys at position 148, a Iso at position 168, and any combination thereof and having anticoagulant effect, vasodilatoryeffect or a combination thereof, or
(vii) a polypeptide comprising a fragment of any of the foregoing wherein an antibody raised against the fragment specifically binds to a procalin 1 polypeptide.

3. A polynucleotide encoding a polypeptide of claim 1 or 2.

4. A polynucleotide comprising SEQ ID NO:1 or 3.

5. A polynucleotide comprising:
at least about 17-60 contiguous nucleotides comprising
SEQ ID NO:1 or 3 and containing nucleotides 76-78, 85-87, 442-444, or 502-504 of SEQ ID NO:1 or 3.

6. A composition comprising a polypeptide of claim 1 or 2 and a pharmaceutically acceptable carrier.

7. An immunogenic composition comprising a polypeptide of claim 1 or 2 and at least one adjuvant.

8. A method of desensitizing a subject to a Triatoma antigen comprising contact the subject with a composition of claim 6.

9. A substantially purified antibody that specifically binds to a procalin variant 1 or procalin variant 2.

10. A composition comprising the antibody of claim 9 and a pharmaceutically acceptable carrier.

11. A method of identifying a source of an anaphylactic reaction comprising contacting a sample from a subject having an anaphylactic reaction with (i) a polypeptide or peptide comprising a sequence as set forth in SEQ ID NO:2 or 4, or (ii) an antibody that specifically binds to a polypeptide encoded by a sequence as set forth in SEQ ID NO:1 or 3.

12. A kit for detecting the presence of anaphylactic antibodies (IgE) in a sample comprising a polypeptide of claim 2 or 4, or polypeptides encoded by polynucleotides of claim 1 or 3.

13. A kit for detecting the presence of a an anaphylactic antigen comprising a substantially purified antibody that specifically binds to a polypeptide of SEQ ID NO:2 or 4.
